(19) United States
(12) Patent Application Publication
(10) Pub. No.: US 2004/0203084 A1
(43) Pub. Date: Oct. 14, 2004

(54) Profiling conformational variants, antibody compositions and methods of using the same

(76) Inventor: Doug Levinson, Sherborn, MA (US)

Correspondence Address: TRANSFORM PHARMACEUTICALS, INC., 29 HARTWELL AVENUE, LEXINGTON, MA, 02421, US

(21) Appl. No.: 10/821,103

(22) Filed: Apr. 8, 2004

Related U.S. Application Data
(60) Provisional application No. 60/461,753, filed on Apr. 10, 2003.

(51) Int. Cl. G01N 33/537; G01N 33/543; G01N 33/53

(52) U.S. Cl. 435/7.92

(57) ABSTRACT

A method for producing a pharmaceutical composition is described. This method comprises providing a plurality of isolated conformational variants and identifying a conformational variant that binds to a neutralizing antibody or raises neutralizing antibodies in vivo. Compositions produced according to the method of the invention are also described.

### RELATED APPLICATIONS

This application claims priority to U.S. Provisional Application No. 60/461,753 filed Apr. 10, 2003 which is hereby incorporated by reference.

### FIELD OF THE INVENTION

This invention provides pharmaceutical compositions comprising protein antigens that elicit a neutralizing antibody response in a subject and methods for producing such pharmaceutical compositions. Such pharmaceutical compositions may be used as compositions for stimulating an immune response including vaccines.

### BACKGROUND OF THE INVENTION

Proteins comprise linear polymers (termed polypeptides) of amino acids. Twenty chemically distinct amino acids are naturally found in proteins. Many of the bonds in a long polypeptide chain allow free rotation of the atoms they join, giving the polypeptide great flexibility. In principle then, a protein molecule could adopt an almost unlimited number of shapes or conformations. For example, a protein of 40 residues can theoretically adopt 10⁴⁰ possible conformations. Most polypeptide chains however, fold rapidly into only one or a few particular conformations, which together are termed the native conformation.

Four levels of protein structure are recognized. Primary structure is the amino acid sequence. Secondary structure refers to the regular three-dimensional arrangement of amino acid residues that are close to one another in the linear sequence; i.e., it is the local conformation of the polypeptide backbone. Tertiary structure refers to the conformation of the protein i.e. the spatial arrangement of amino acid residues that are far apart in the linear sequence and to the pattern of disulphide bonds. In short, the folding of a protein's secondary structural elements together with the spatial position of its side chains composes the tertiary structure. Quaternary structure refers to the spatial arrangement of subunits or tertiary structures and the nature of their contacts. For example, quaternary structure is when some proteins consist of several polypeptide chains, arranged in a regular or ordered manner.

Most proteins fold spontaneously into their native conformation. By treatment with certain reagents, a protein can be unfolded or denatured to give a flexible polypeptide chain that has lost its native conformation. When the denaturant is removed, the protein can refold spontaneously into its original conformation, indicating that all the information necessary to specify the shape of a protein is contained within the amino acid sequence itself.

Folding occurs because the amino acids interact with one another and with water to form various favorable and weak non-covalent bonds and electrostatic interactions. The hydrophobic side chains of the non-polar amino acids tend to be pushed together in the interior of the molecule, which enables them to avoid contact with the aqueous environment. By contrast the side chains of the polar and charged amino acids tend to arrange themselves near the outside of the protein molecule where they can interact with water and other polar or charged molecules. Since the peptide bonds are themselves polar, they tend to interact both with one another and with polar side chains to form hydrogen bonds. Nearly all polar residues buried within the protein are paired in this way. Hydrogen bonds thus play a major part in holding together different regions of a polypeptide chain in a folded protein molecule.

Many proteins including secreted or cell surface molecules often form additional covalent intrachain bonds. For example, these proteins may form disulphide bonds between two —SH groups of neighbouring cysteine residues, which serves to further stabilize the three dimensional structure of extracellular proteins. These bonds are not required for the specific folding of proteins, since folding occurs in normally in the presence of reducing agents.

The net result of all the individual amino acid interactions is that most protein molecules fold up spontaneously into precisely defined conformations. The positioning and chemistry of the different atoms on the surface of a protein in its native conformation determine the ability of the protein to bind to other proteins, including antibodies.

The immune system recognizes the structure of foreign antigens, many of which are proteins, and mounts an immune response. There are two broad classes of immune responses: cell-mediated responses and antibody responses. Cell-mediated immune responses are mediated by T-cells. Receptors present on these T-cells recognize an antigen that has been unfolded, degraded or otherwise processed and displayed on the surface of other host cells. The T-cells can either kill the host cell presenting the antigen or secrete chemical signals that activate macrophages to destroy the invading microorganisms.

Antibody responses are mediated by B-cells and involve the secretion of antibodies which bind specifically to the foreign antigen that induced them. These antibodies frequently recognize and bind to conformational epitopes that may be composed of amino acids that are discontigous in primary sequence but which are brought into close proximity in three dimensional space. Such conformation dependent epitopes are commonly referred to as topographical determinants or conformational epitopes (see Berzofsky and Berkower, Immunogenicity and Antigen Structure in Fundamental Immunology, Fourth Edition, Ed. William Paul, 1999). The antibodies secreted inactivate viruses or bacterial toxins by blocking their ability to bind to receptors on host cells. Antibody binding also marks invading microorganisms for destruction either by making it easier for a phagocytic cell to ingest them or by activating a system of blood proteins collectively called complement that kills the invaders. The antibody response is also known as the humoral response.

The process of vaccination harnesses the immune system to protect a subject from invading microorganisms. In vaccination, the body is immunized with vaccines comprising antigens that stimulate the formation of antibodies that will protect against the disease. For example, dead organisms are injected to protect against bacterial diseases such as typhoid fever and whooping cough, attenuated toxins are injected to protect against tetanus and botulism and attenuated organisms are injected to protect against viral diseases including poliomyelitis and measles.

In order for the vaccine to generate a protective immune response, the vaccine preparation must be immunogenic. This means that the antigen must be capable of inducing an immune response. Some agents such as tetanus toxin are innately immunogenic and may be administered in vaccines without modification. Other important agents are not immunogenic and must be converted into immunogenic molecules before they can induce an immune response. This may be done by using an adjuvant and most conventional vaccines comprise an adjuvant to enhance the immune response to the antigen.

An adjuvant is a substance which non-specifically enhances an immune response to an antigen in a host organism. Adjuvants are thought to mediate their effects in three ways. First, adjuvants may adsorb or precipitate the antigen and retain it at the site of injection. This prevents the antigen from being removed and degraded by the body allowing the antigenic stimulus to continue over a prolonged period of time. Adjuvants which work by this mechanism are known as depot antigens, and examples are Freund's complete adjuvant, aluminium phosphate and alumunium hydroxide. Depot adjuvants often provoke severe persistent local reactions such as granulomas, abscesses and scarring when injected subcutaneously or intramuscularly.

Second, adjuvants may act to deliver the antigen to the spleen and/or lymph nodes where the cell interactions that lead to the development of plasma cells (antibody secreting cells) take place. Adjuvants may achieve this by stimulating endocytosis of the antigen by antigen presenting cells (e.g. macrophages, dendritic cells, B-lymphocytes). Antigen presenting cells “loaded” with antigens migrate to the lymph nodes via the lymphatic system.

Third, adjuvants may directly or indirectly activate the various cells involved in the immune response.

Microparticle adjuvants are known in the art. Such particles present multiple copies of a selected antigen to the immune system and promote endocytosis of antigens by antigen presenting cells, thereby stimulating both humoral and cell mediated immunity.

U.S. Pat. No. 6,458,370 summarises prior art methods of producing biodegradable polymeric microparticles. Vaccine compositions comprising microparticles with entrapped or adsorbed antigens in combination with submicron oil-in-water emulsions are disclosed.

U.S. Pat. No. 6,565,777 describes a method for producing biodegradable polymeric microparticles which results in particles of generally less than 10 mm in diameter. The method is capable of producing microparticles on an industrial scale.

U.S. Pat. No. 5,753,234 discloses microparticles comprising a core of protein antigen coated with a biodegradable polymer, which degrades slowly in vivo, releasing the antigen over a period of time.

U.S. Pat. No. 6,534,064 discloses the production of stable protein particles formed by chemical precipitation, by the use of chemical cross linking agent or by heat stabilization, and the use of such protein particles in vaccines. The protein contained in such protein particles is denatured and may not induce an effective B-cell response.

U.S. Pat. No. 6,355,271 discloses calcium phosphate particles of between about 300 nm and about 4000 nm, and methods of producing these particles. Vaccines comprising antigens adsorbed to the surface of these particles are described.

U.S. Pat. No. 5,942,242 discloses the use of ion exchange resins or adsorbant resin powders in vaccines.

U.S. Pat. No. 6,585,973 discloses the use of solid phase adjuvants in combination with antigens and carbohydrates. The solid phase adjuvants described include aluminium and calcium salts.

Preferably a vaccine generates a neutralizing antibody response. A neutralizing antibody is one that decreases the infectious titre of a preparation of the invading microorganism. The neutralizing power of a serum usually reflects the degree of protection in an infected animal.

Neutralizing antibodies raised against viral antigens appear to interfere with a step leading to the introduction of the viral genome into a host cell. For example, the b12 antibody recognizes gp120, the surface envelope glycoprotein of HIV1 that binds to the CD4 molecule on the surface of host cells. The binding of the b12 antibody is thought to interfere with the virus binding and introducing its nucleic acid into host cells.

The majority of vaccines currently available do not selectively or preferentially induce neutralizing antibodies. Vaccines which do not selectively induce neutralizing antibodies provide a variable level of immunity against infection. To date, however, there is no method of producing vaccines that selectively induce neutralizing antibodies and provide a consistently high level of protection.

A need thus exists for a method for producing vaccines which reliably elicit a strong neutralizing response.

It is well known in the art that recombinant protein vaccines tend to have low efficacy as compared to other vaccines types such as attenuated live virus or conjugated vaccines. This may be due in part to the inability of the recombinant protein to generate both robust cellular and neutralizing humoral responses. It is desirable therefore that recombinant protein vaccines be designed such that they provoke both types of host immune responses.

### SUMMARY OF THE INVENTION

In one embodiment, the present invention relates to a method of producing a composition comprising a protein antigen, said method comprising the steps of providing a plurality of samples comprising a protein, said samples differing with respect to the conformational state of said protein; identifying a sample from said plurality of samples that comprises a conformational variant of said protein capable of stimulating the production of neutralizing antibodies against a pathogen from which said protein was derived.

In another embodiment, said conformational variants have the same primary amino acid sequence, but differ with respect to their secondary or tertiary structure.

In another embodiment, said method comprises a step of clustering samples based on a profile of selected criteria.

In another embodiment, said criteria comprises the ability to bind, or the affinity, for an antibody or plurality of antibodies specific for said protein.

In another embodiment, said antibody is: a non-neutralizing antibody; a neutralizing antibody; a polyclonal antibody; or a monoclonal antibody.

In another embodiment, said antibody is contacted with said protein in the presence of biological fluids.

In another embodiment said conformational variants are obtained by treating: said protein under different conditions; a sample of native protein under different conditions; a sample of denatured or partially denatured protein under different conditions; or by stabilizing a secondary or tertiary structure of said conformational variant.

In another embodiment, said identifying comprises identifying a sample comprising a protein-conformational variant that: binds to a neutralizing antibody; binds to a neutralizing antibody with higher affinity than the neutralizing antibody binds the native conformation; bind to one or more neutralizing antibodies with a higher relative binding affinity than one or more non-neutralizing antibodies; at least partially clears neutralizing activity in serum in vitro; clears neutralizing activity in serum in vitro to a greater degree than the same protein in a native conformation; or stimulates the production of neutralizing antibodies in vivo to a greater degree or more potently than the same protein in a native conformation.

In another embodiment, said protein is structurally characterized using method selected from the group consisting of circular dichroism spectropolarimetry, fluorescence spectroscopy and its derivatives using either intrinsic or extrinsic fluorescent probes, mass spectroscopy, UV-VIS spectroscopy, NMR, small angle X-ray scattering, enzymatic activity, protease digestion mapping, hydrogen-deuterium exchange methods employing mass spectroscopy or NMR, ion exchange chromatography, hydrophobic interaction chromatography, reverse phase chromatography, gel filtration chromatography and affinity chromatography.

In another embodiment, said conformational variant is stabilized using a covalent linker.

In another embodiment, said covalent linker targets an amino group, a carboxyl group, a hydroxyl group, a guanidinyl group, a sulfhydryl group or a carbohydrate.

In another embodiment, said linker target is created by in vitro mutagenesis of the amino acid sequence or a nucleic acid sequence encoding said protein.

In another embodiment, said linker target is created by in vitro modification of primary amines using 2-Iminothiolane, N-Succinimidyl S-acetylthioacetate or N-Succinimidyl 3-[2-pyridyldithio]propionate.

In another embodiment, a purification step enriches a sample for a conformational variant.

In another embodiment, said sample is enriched for a conformational variant using ion exchange chromatography, hydrophobic interaction chromatography, reverse phase chromatography, gel filtration chromatography or affinity chromatography.

In another embodiment, a said sample is enriched using one or more neutralizing antibodies.

In another embodiment, said affinity is measured using a method selected from the group consisting of: ELISA, surface plasma resonance, gel mobility shift assay, isothermal titration calorimetry, centrifugation and fluorescent resonant energy transfer.

### BRIEF DESCRIPTION OF THE DRAWINGS

FIG. 1 shows schematically results of profiling a number of conformational variants with a panel of monoclonal antibodies. The angle of the line in each sample box represents the relative binding affinity of an antibody for a sample.

FIG. 2. HPLC chromatograms of Bovine Pancreatic Trypsin Inhibitor (BPTI) cross linked and quenched after the indicated refolding intervals under oxidative conditions. The buffer system was 20 mM phosphate, pH 6.2, 25° C.

FIG. 3. Relative population of the native state of BPTI in the presence of unary and binary mixtures of dithiothreitol (DTT) and trifluoroethanol (TFE). The buffer system was 10 mM phosphate, 0.2 mM EDTA, pH 7.6, 25° C.

### DETAILED DESCRIPTION OF THE INVENTION

Accordingly, in a first aspect, the present invention provides a method for producing a pharmaceutical composition, which method comprises a step of providing a plurality of isolated conformational variants of a protein and identifying a sample of samples comprising a conformational variant or subset of variants capable of stimulating the production of neutralizing antibodies in vivo. The method may include a step of producing conformational variants or a step of stabilizing said conformational variants. The method may further comprise the steps of profiling the samples between or among antibodies, e.g., based on a set of criteria, such as relative binding affinity between neutralizing and non-neutralizing. The method may further include a step of clustering samples, or a conformational variant or subsets of conformational variants within a sample with a similar profile of selected criteria. The method may further comprise a step of binding a conformational variant to a microparticle, preferably a microparticle having a diameter of up to 150 μm. Further included are compositions produced by the methods of the present invention. The method may further include a step of enriching a sample for a conformational variant. The method may further include a step of identifying samples containing conformational variants that will bind neutralizing antibodies or will clear neutralizing activity in vivo.

A composition of the present invention may be a composition capable of stimulating an immune response against conformational variants of the protein. Preferably, the immune response comprises generating antibodies against a protein or peptide. Preferably, the antibodies are neutralizing antibodies. Advantageously, a vaccine produced according to the present method reliably elicits a neutralizing response. Preferably, the compositions are pharmaceutical compositions. Preferably the conformational variant is more potent or more efficacious than same protein in a native conformation for stimulating an immune response, raising or generating neutralizing antibodies, or another activity disclosed herein.

Each step of the method of the present invention will now be discussed in greater detail.

Provision of conformational variants: The first step in the method for producing a composition of the present invention is the provision of a plurality of isolated conformational variants of a target protein. Conformational variants are proteins having all or a portion of the same primary amino acid sequence, but having different tertiary or secondary structures or conformations. As used herein the term “protein” includes both full length polypeptides and portions thereof. The protein may be wild type, mutant or otherwise modified. A limitation is that the protein is able to assume multiple conformations depending on the environmental conditions. Preferably, the protein comprises a primary sequence capable of giving rise to at least two distinct secondary structures, e.g., alpha helix or beta sheet. The term “antigen” as used herein is a substrate capable of causing the production of an antibody, preferably in vivo.

Any protein may be used as a target protein. Preferably however, the target protein used to generate conformational variants is an antigen capable of binding to a neutralizing antibody, such as HIV gp120, anthrax protective antigen, botulism toxin, enterotoxin B, SARS spike protein, influenza hemagglutinin, respiratory syncytial virus (RSV) attachment and fusion proteins Yersinia outer proteins, measles virus, haemagglutinin, small pox surface and attachment proteins, diphtheria toxin, tetanus toxin, pertussis toxin, and ebola surface and attachments proteins. In one embodiment, the majority of antigen does not normally (less than 75% of the time) assume a conformation necessary for binding a neutralizing antibody or does not normally (less than 75% of the time) assume the necessary conformation for binding a neutralizing antibody.

The conformational variants of a target protein may share the primary sequence of amino acids of the wild type target protein, or the amino acid sequence of a mutant version (naturally occurring or man-made) of the protein, or a derivative thereof. Preferably the protein is from a virulent pathogen (e.g., virus, bacterium or fungus). In one embodiment, the protein is responsible or partially responsible for the pathogen's virulence or disease.

Where the amino acid sequence of the conformational variants is that of a mutant version of a protein, it is preferred that the amino acid sequence is substantially identical to that of the wild type protein. Preferably, the degree of identity with the native protein is 85% or higher. More preferably, the degree of identity is 90% or higher, 95% or higher, or 99% or higher. The mutations present in the amino acid sequence may be naturally occurring mutations. In one embodiment, the mutation is the cause of or increases the virulence of the pathogen. Alternatively, the mutations may be generated in protein produced by recombinant methods using the technique of in vitro mutagenesis of the nucleic acids encoding the protein. Typically, the protein may be mutated using in vitro techniques to replace amino acid residues with cysteine or lysine residues, or to add cysteine or lysine residues. Conveniently, in vitro mutagenesis is performed using Stratagene's Quikchange® site directed mutagenesis kit, following the instructions of the manufacturer. In one embodiment, the amino acid sequence is the sequence of a pathogenic form of the organism of interest.

A plurality of conformational variants of a target protein (also known as a library of conformational variants) may be obtained by treating a plurality of isolated samples of the target protein under different conditions to generate multiple conformational variants of the target protein. Each treated sample may contain a single or multiple conformational variants.

In one embodiment for producing conformational variants, isolated samples of the target protein comprise native protein. These samples of native protein are treated under different conditions. Conditions which may be varied include the composition of the buffer, the time allowed for treatment, and temperature. For example, at least one of the samples of target protein in its native conformation may be treated with a denaturing agent or treated under denaturing conditions. Suitable denaturing agents include organic solvents, pH modifiers, chaotropes, ionic detergents, oxidizing agents, and precipitants. Chaotropic denaturing agents include urea and guanidinium salts such as guanidinium hydrochloride or guanidinium isothiocyanate. pH modifiers include acids and bases. Ionic detergents that act as denaturing agents include sodium dodecyl sulfate, sodium deoxycholate, sodium taurocholate and glycocholic acid, sodium salt. Oxidizing agents include β-mercaptoethanol, 1,4 dithiothreitol, dithrioerythitol, and Tris[2-caroxymethyl]phosphine hydrochloride. Precipitants that act as denaturing agents for certain target proteins include ammonium sulfate. Denaturing conditions include sonication, temperatures of 40° C. or higher, or temperatures of 0° C. or lower, or pressure.

In another embodiment for producing conformational variants, the isolated samples of the target protein comprise denatured or partially denatured protein. The protein may have little or no secondary or tertiary structure. Structure determining techniques may optionally be used to determine whether the protein is in a denatured or partially denatured conformation. Suitable techniques include NMR, circular dichroism spectroscopy, calorimetry, native polyacrylamide gel electrophoresis, gel filtration, fluorescence spectroscopy, ultraviolet-visible spectroscopy, Raman spectroscopy, small angle X-ray scattering, or loss of activity in an assay measuring a function of the native protein.

Where the target protein is present in a denatured or partially denatured form, isolated samples of the protein may be treated under a variety of conditions to generate conformational variants. Conditions which may be varied include the composition of the buffer, the time allowed for treatment, the temperature, and the pressure. For example, a denaturing agent may be removed from the samples of denatured protein, or the samples of denatured protein may be treated with a renaturing or stabilizing agent i.e. an agent which restores at least partially secondary or tertiary structure, or the pH may be altered.

The treatment of isolated samples of the target protein may be carried out in parallel (typically in a multiwell plate). Therefore, this step has a high throughput capability.

In a preferred embodiment, the structure of each isolated conformational variant or subset of the target protein is stabilized. A suitable stabilization method comprises the formation of intermolecular or intramolecular covalent bonds. An intramolecular bond involves only functional groups of a single protein molecule. An intermolecular bond involves at least one other protein molecule. An intramolecular bond includes situations where a second molecule bridges a bond between two functional groups within the same protein, e.g., a covalent cross-linking agent. Other stabilization methods comprise the formation of inter- or intramolecular hydrogen or ionic bonds.

In one embodiment, non-native intermolecular covalent bonds are formed by treatment of the conformational variants of the target protein with a chemical cross-linking agent. In the context of this application, a chemical cross-linking agent is a molecule with at least two reactive groups that are capable of forming covalent cross-links in protein molecules. At least part of the chemical cross linking agent is integrated into the protein as a cross-link. Suitable chemical cross-linking agents for use in this invention include, but are not limited to, formaldehyde, glutaraldehyde, glyocal, malondialdehyde, succinialdehyde, adipaldehyde, phthalaldehyde, 4-Azidobenzoic acid (3-sulfo-N-succinimidyl) ester sodium salt, 1,4-Bis [3-(2-pyridyldithio) proprionamido]butane, Bis [2-(N-succinimidyl-oxocarbonyloxy)ethyl] sulfone, Bis-maleimidohexane, Bis-[2-(4-azidosalicylamido) ethyl] disulphide, 1,5-Difluoro-2,4-dinitrobenzene, Dimethyl pimelimidate, Dimethyl 3-3′-dithioproprionimidate dihydrochloride, Ethylene glycol disuccinate di(N-succinimidyl) ester, N-hydroxysuccinimidyl-4-azidosalicylic acid, and Sebacic acid bis(N-succinimidyl) ester. These chemical cross-linking agents are available for example, from Sigma Aldrich and Pierce Biotechnology, Inc.

Intramolecular covalent bonds may be formed for example, by exposure to conditions that induce the formation of covalent crosslinks. Conditions that induce the formation of covalent crosslinks include oxidizing conditions, UV irradiation and pH.

Where the protein has two or more cysteine residues, oxidising conditions may induce the formation of intramolecular disulphide links (which are formed between the thiol groups of two cysteine residues).

In one embodiment, the cross linking reaction is performed under equilibrium conditions. In this case, a native protein is partially denatured under reducing conditions and allowed to equilibrate. Buffer components or additives which may interfere with the cross linking reaction are then removed by dialysis, desalting or other appropriate methods. A covalent cross linker or combination of cross linkers are then crosslinked to the protein using appropriate conditions. Variables that can be optimized include protein to cross linker ratios, cross linker spacer arm length, temperature, pH, ionic strength, time of reaction, and quenching method.

In another equilibrium embodiment, unfolded protein is partially renatured under reducing conditions and allowed to equilibrate. Buffer components or additives which may interfere with the cross linking reaction are then removed by dialysis or desalting methods. A covalent cross linker or combination of cross linkers are then crosslinked to the protein using appropriate conditions. Variables to be optimized include protein to cross linker ratios, cross linker spacer arm length, temperature, pH, ionic strength, time of reaction, and quenching method.

In another embodiment, the protein is denatured under reducing conditions and allowed to equilibrate. Oxidative refolding is then initiated by the removal or dilution of the denaturant in the presence of an oxidizing agent. Suitable oxidizing agents include oxidized dithioβ-mercaptoethanol, oxidized 1,4 dithiothreitol, oxidized dithrioerythitol, oxidized Tris[2-caroxymethyl]phosphine hydrochloride, ozone, oxygen, hydrogen peroxide, peroxyacids, oxidized glutathione, and halogens. At specific time intervals, a covalent cross linker or combination of cross linkers are then crosslinked to the protein using appropriate conditions.

In another embodiment, unfolded protein is equilibrated under reducing conditions. Oxidative refolding is then initiated by the removal or dilution of the denaturant in the presence of an oxidizing agent. Suitable oxidizing agents include oxidized dithioβp-mercaptoethanol, oxidized 1,4 dithiothreitol, oxidized dithrioerythitol, oxidized Tris[2-caroxymethyl]phosphine hydrochloride, ozone, oxygen, hydrogen peroxide, peroxyacids, oxidized glutathione, and halogens. At specific time intervals, a covalent cross linker or combination of cross linkers are then crosslinked to the protein using appropriate conditions.

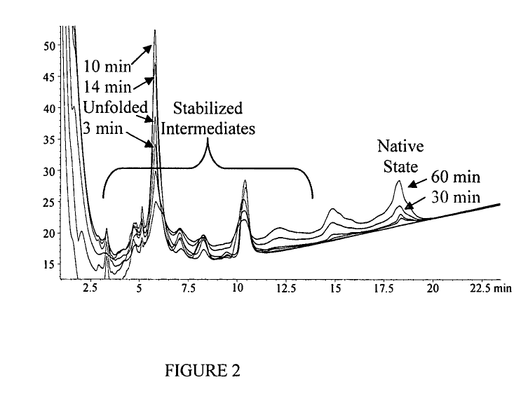

Conformational subsets or species can be further isolated for a sample(s) by single or multiple chromatographic steps (see FIG. 2). Suitable techniques include ion exchange chromatography, hydrophobic interaction chromatography, reverse phase chromatography, gel filtration chromatography, isoelectric focusing, and gel electrophoresis. Conformational variants may then be further characterized by selected criteria including but not limited to: circular dichroism spectropolarimetry, fluorescence spectroscopy and its derivatives using either intrinsic or extrinsic fluorescent probes, mass spectroscopy, UV-VIS spectroscopy, NMR, small angle X-ray scattering, enzymatic activity, protease digestion mapping, and hydrogen-deuterium exchange methods employing mass spectroscopy or NMR.

Identification of protein antigens that bind or give rise to neutralizing antibodies: The method may comprise contacting each conformational variant sample with a neutralizing antibody, assaying the samples for their ability to clear neutralizing activity in vitro or assaying the samples for their ability to give rise to neutralizing antibodies in vivo. It is noted that each sample may contain more than one or a sub-population (sub-set) of conformational variants. For samples with subpopulations, it may be desirable to perform iterative experimentation using conditions similar to the conditions giving rise to the subpopulation to identify conformers and conditions that give rise to a specific conformer. As discussed herein, samples may be enriched for one or more conformational variants. Neutralizing epitopes are frequently conformational epitopes due to the fact that they are often the site of the antigen that is required for interaction with host protein.

Samples are assayed to determine those which posses an increased apparent affinity (or association constant, Ka) to one or more neutralizing antibodies. It is important to note that Ka reflects 1) the intrinsic binding activity of the antigen to the antibody and 2) the relative proportion of the antigen that is in the conformation that is recognized by the specific antibody. This relationship has been described (see Berzofsky and Berkower in Immunogenicity and Antigen Structure in Fundamental Immunology, Fourth Edition, Ed. William Paul, 1999). It is desirable to find conditions that produce conformational populations with increased apparent association constants as compared to the native state. It is expected that using the techniques disclosed herein will identify populations that have an increased proportion of protein in the conformation that is recognized by a neutralizing antibody. Conformational populations that possess increased association constants (relative to either their native or denatured states) will either possess 1) an increased intrinsic affinity between antigen and neutralizing antibody or 2) an increased proportion of protein in the conformation that is recognized by a neutralizing antibody, or both. Either way to increase the apparent affinity of antigen to neutralizing antibody is desirable and may result in improved neutralizing potential of the antigen.

Increased apparent affinity of conformational populations may result from the interaction of antigen with polyclonal antibody preparations (or sera) that contain neturalizing activity. In this case, a change in conformational state may preferentially bind one or more specific antibodies that possess increased binding affinities. Alternatively, the relative proportion of the antigen that is in a conformation that is specifically recognized by one or more antibodies in the antibody population.

The samples may be assayed to determine which conformational variants bind to a neutralizing antibody as a protein antigen. Typically, each conformational variant sample is contacted with a neutralizing antibody under substantially identical conditions. In this context, the phrase substantially identical conditions generally means that the composition of buffers, the incubation times, the pressure, and temperatures used in the reaction to determine the affinity of each conformational variant for the profiling protein are similar or do not significantly affect binding affinity. The degree of similarity required between the affinity determining reactions may be varied depending on the needs or desires of the user.

The step of contacting each conformational variant with the neutralizing antibody may advantageously be determined in the presence of biological fluids (e.g., blood, semen, plasma, proteins, etc.) and/or adjuvants since these may affect the structure of conformational variants.

In this application, the word antibody encompasses polyclonal antibodies, monoclonal antibodies, single chain antibodies, chimeric antibodies, fragments derived from proteolysis of whole antibodies and/or by reduction of disulphide bonds, or antibodies generated by means of expression libraries, e.g., phage display. A “neutralizing antibody” as used herein is defined the same as its normal use in the field of immunology, specifically, an antibody that inhibits or blocks an infectious or pathologic agent at some point in its pathologic process. For example, a neutralizing antibody may block a virus from cell binding and entry. A neutralizing antibody is an antibody that exhibits neutralizing activity in a standard assay. In one embodiment, the neutralizing antibody is capable of precipitating antigen from an aqueous solution containing the antigen.

The “affinity” or “degree of binding” of the conformational variants for the neutralizing antibody may be measured by standard methods. Suitable methods include surface plasmon resonance (using, for example, a BIAcore® S51 machine in accordance with the manufacturer's instructions), an ELISA assay (where the profiling protein is an antibody), plasmon waveguide resonance (using, for example, a Proterion PWR spectrophotometer used in accordance with the manufacturer's instructions), isothermal titration calorimetry, analytical ultracentrifugation, immunoprecipitation, or affinity chromatography (possibly employing phage display clones in the affinity matrix).

The term “affinity” as used herein includes both the true or microscopic affinity and apparent affinity, but the term may be specified as one or the other. The true or microscopic affinity (Kmicro) is dependent on the rate constants for binding and unbinding (kon and koff, respectively): Kmicro=koff/kon, whereas the apparent affinity (Kapp) is a function of both the microscopic affinity and the number of sites (N) present in the sample: Kapp=Kmicro/(2^{1/N}−1). In the case of the present invention, the true and apparent affinities will be the same if a single conformation has a single antibody binding site. Apparent affinities are measured when there are other conformations present which have different microscopic affinities. For example, if there are 2 conformations of which an antibody can only bind to one, the apparent affinity measured in an experiment will depend on both the microscopic affinity of the antibody for that conformation and the equilibrium constant (Keq) governing the amount of that conformation (i.e., Kapp=Kmicro*Keq).

Conformational variants that bind to a neutralizing antibody are identified as protein antigens. Preferably, the conformational variant has a high apparent affinity for the neutralizing antibody. Alternatively or in addition thereto, the samples are tested to determine whether they can raise neutralizing antibodies in vivo.

Because the protein antigen identified by this method binds to a neutralizing antibody, it possesses an epitope that is recognized (to some degree) by a neutralizing antibody. Therefore, the protein antigen may elicit the production of antibodies which recognize the same epitope. Such antibodies would be expected to possess neutralizing activity.

Preferably, a conformational variant sample of the target protein is additionally contacted with a non-neutralizing antibody and a neutralizing antibody that recognizes the target protein, and a protein antigen is identified that binds to the neutralizing antibody with a higher affinity than to the non-neutralizing antibody. Conformational variants that bind to a neutralizing antibody with higher affinity than to the affinity between the neutralizing antibody and the native conformation antibody present an epitope in a manner that is readily accessible to and recognized by the neutralizing antibody. Since they easily present this epitope, they are better able to elicit a neutralizing response. Preferably, a conformational variant sample will have a greater increase in affinity for a neutralizing antibody over the native conformation as compared to any increase in affinity of a non-neutralizing antibody for the conformational variant over the native conformation. Preferably, the affinity of a neutralizing antibody for a conformational variant will increase and the affinity of a non-neutralizing antibody will decrease, as compared to the native conformation. In addition, these conformational variants that are protein antigens may not present or possess other epitopes (recognized by non-neutralizing antibodies) that are not essential to the function as a protein antigen for neutralizing antibodies and will not elicit the production of non-neutralizing antibodies.

For example, a conformational variant of gp120 that binds to the b12 antibody possesses the epitope recognized by this antibody. This variant may elicit production of antibodies sharing the neutralizing properties of the b12 antibody but would not elicit production of non-neutralizing antibodies. The assays for determining whether a conformational variant sample will bind a neutralizing antibody, give rise to neutralizing antibody, or preferentially binds to a neutralizing antibody can be done before or after conformational variant sample clustering.

In one embodiment, the relative binding affinity between antibodies is determined using antibodies that bind the same epitope.

Profiling conformational variant samples of a target protein and clustering of conformational variant samples: The conformational variants may be profiled based on selected criteria, included chemical, physical and functional characteristics. In one embodiment, the conformational variants are profiled based on their binding affinity (actual or relative) to one or more antibodies. In this embodiment, the conformational variant samples are contacted with an antibody and the binding affinity of the conformational variants for this antibody is determined by methods standard in the art. The samples may be purified or partially purified to enrich the sample for one or more specific conformations before or after profiling. The affinities of each conformational variant for the antibody are preferably determined under substantially identical conditions. Once the conformational variants are profiled, they can be clustered based on the similarity of their profiles. As discussed, the degree of similarity required for two or more conformational variants to be in the same cluster would be clear to one skilled in the art for the specific method used. However, a suitable guide for assessing similarity would be to ensure that the incubation times, the temperatures of the reaction, the atmospheric pressure, and the concentration of each component of buffers common to each reaction should not vary by more than 10%. More preferably, the degree of variation should be 5% or less or within the normal range for the assay used. It is also permitted that chemically similar compounds may be substituted for one another in buffers.

As discussed herein, those conformational variants which bind to each antibody with a similar affinity are classified into the same cluster. Conformational variants having substantially different affinities for each antibody are assigned to different clusters. The similarity required in the binding affinities to classify two conformational variants as belonging to the same cluster will depend upon the specific criteria of the user but is reproducible when using the same criteria.

Typically, two or more measurements of the affinity of each conformational variant or subset for each antibody are taken. A mean binding affinity of each conformational variant for each antibody may then be determined. In addition, the standard error of the mean may be calculated using the formula σ/√N, where σ is the standard deviation and N is the number of readings taken.

For the affinities of two conformational variants for an antibody to be considered similar, the mean calculated affinities should be within three standard errors of any of the conformational variants. More preferably, the mean binding affinities should be within two standard errors. More preferably, the mean binding affinities should be within a single standard error. Other criteria may also be used to determine whether two conformational variants belong to the same cluster.

Ideally, the profiling or clustering process should result in the conformational variants being grouped into multiple profiles. In an ideal situation, the number of profiles is significantly less than the number of conformational variants, but greater than 1. To ensure that conformational variants or subsets are grouped together in a useful way that reflects the similarity in properties of the conformational variants or subsets, the profiling process may be modified as appropriate. For example, the skilled person may initially assign all the conformational variants or subsets whose mean affinities are within three standard errors of the mean affinities of other conformational variants or subsets to a single profile. If numerous profiles are identified, no further attempt would be made to subdivide the profiles. It is considered that there are numerous profiles if the number of profiles identified is 50% or more than the number of conformational variants tested.

If the number of clusters identified is less than 50% of the number of conformational variants, the clusters may be subdivided. Those conformational variants whose mean affinity for the antibody falls within two standard errors of the mean affinities of other conformational variants should thus be identified. The conformational variants whose mean affinities are within this range are assigned to the same cluster. If numerous profiles are identified by this mechanism, no attempt is made to further classify the conformational variants. If this is not the case, profiles may be determined which comprise conformational variants having affinities that are within a single standard error of each other.

Where two or more antibodies are used, the same method is used to assign each conformational variant sample into groups based upon the affinity of the variant for the first antibody and upon the affinity of the variant for the second (and subsequent) antibody. The variants or subsets are then assigned to clusters. Each cluster comprises variants or subsets that fall into the same groups for all antibodies used.

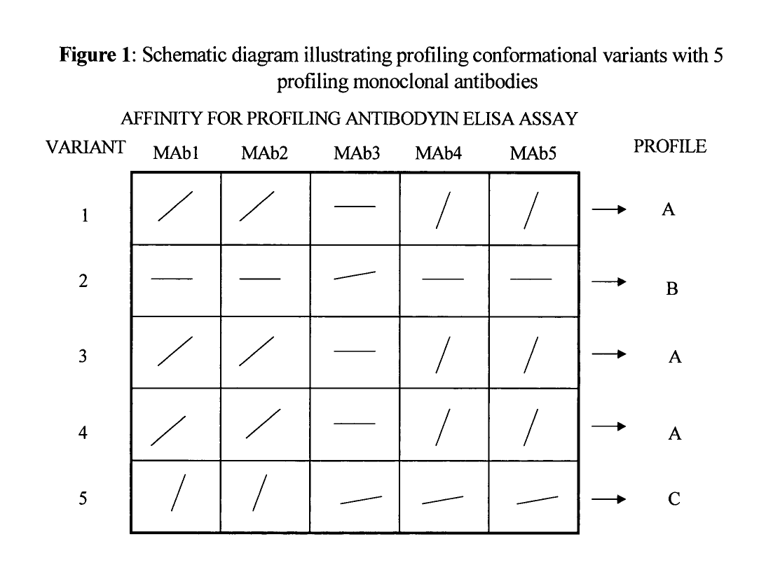

The profiling process is illustrated (schematically) in FIG. 1. FIG. 1 shows the results of several ELISA assays. ELISA assays are performed using five different monoclonal antibodies and five different conformational variants samples of a protein. Those conformational variants having a similar pattern of binding to all five antibodies and thus, each having a similar profile are grouped into the same cluster. Three profiles are identified in this case. Profile A includes those conformational variants or subsets that recognize monoclonal antibodies 1, 2, 4 and 5. Profile B comprises variant or subset 2, which does not significantly recognize any of the profiling antibodies. Profile C comprises variant or subset 5, which strongly recognizes monoclonal antibodies 1 and 2, and weakly recognizes monoclonal antibodies 3, 4 and 5.

As mentioned above, the conformational variants having a similar or same profile preferably have a similar tertiary and secondary structure. To check this, the structure of the conformational variants in each profile may be investigated further. Any suitable technique may be used, although those suited to high throughput are preferred. Suitable techniques include native polyacrylamide gel electrophoresis, calorimetry, circular dichroism spectroscopy, fluorescence spectroscopy, Raman spectroscopy, infrared spectroscopy, X-ray crystallography, protease digestion, activity assays, NMR and gel filtration. Once a profile for each sample is determined based on selected chemical, physical and functional characteristics, the profile is used to cluster samples with similar profiles. Profile data may be utilized in different ways to cluster the samples. Preferably, the clustering is based on one or more of relative binding affinity for multiple antibodies, secondary structure, or tertiary structure.

As discussed, conformational variant samples with a similar profile of selected criteria are clustered. One or representatives of the cluster may be used for further study. Clustering reduces the number of samples undergoing further analysis because only select representatives from each cluster need to be used for further testing. Clustering also allows one to separate different antigens that have similar characteristics, e.g., ability to bind neutralizing antibodies with increased affinity over the native conformation.

In another step, a panel of antibodies is used to cluster conformational variant samples with similar antibody binding profiles. Typically, a panel of antibodies recognizing proteins having the same primary amino acid sequence is used. A panel of antibodies recognizing a (sub)domain of the protein can also be utilized. In one embodiment the antibodies recognize the same epitope. In another embodiment, the antibodies recognize different epitopes. This panel preferably comprises at least one neutralizing antibody. The relative affinities of each antibody for each sample are then measured. Following the determination of the relative affinities with which each conformational variant sample binds to each antibody, profiles of conformational variants can be identified, wherein each member of the profile has a similar pattern of binding. The similarity required in the binding affinities to classify two conformational variants as belonging to the same profile can be determined using various clustering methods known in the art. It is noted that different users may use different clustering methods and thus, some clusters may contain different samples. This is within the present invention as the object is to cluster or separate different samples or groups of samples, not to cluster in one manner.

In particular embodiments of the invention, data from measuring the selected criteria, e.g., relative binding affinities, is processed using what is referred to herein as “clustering” which allows the rapid analysis and identification of similar samples by creating, for example, a family or similarity map. Preferred embodiments of the clustering system comprise a hardware-based instrumentation platform and a software-based suite of algorithms. The computer software is used to analyze, identify and categorize groups of samples having similar properties based on the data obtained from measuring selected criteria, thus identifying a group from which the operator, or scientist, can then select samples for further analysis (e.g., ability to clear neutralizing activity from serum. This selection can be performed independently by the scientist or using an automated means, such as software designed to automatically select samples of interest.

Clustering is generally used in this invention to detect similarities in the properties of a plurality of samples by observing their binning behavior. Thus, the number of interesting conformational variants of a protein antigen can be estimated by binning collected data. For example, the relative binding affinities of a plurality of samples are obtained and used to obtain a binary fingerprint. Advantageously, the affinities (or other data) are compared pairwise between 2 samples in accordance with a metric to generate a similarity score. Other comparisons that use data from more than two samples concurrently are also acceptable, although possibly complex.

One or more clustering techniques can be used to generate bins that are preferably well defined, although this is not an absolute requirement since it is acceptable to generate a reduced list of candidate samples for a given protein antigen as an estimate of the heterogeneity of the conformational variants. Advantageously, the generation of bins facilitates the ready evaluation of conformational heterogeneity among samples.

The invention also encompasses the use of hierarchical clustering to represent the data in the form of a similarity matrix having similar samples listed close together. Such a similarity matrix may be sorted to generate similarity regions along a diagonal. The resulting sorted similarity matrix may be used as a basis for setting the number of clusters for k-means clustering or other clustering techniques based on a specified number of clusters such as Gaussian Mixture Modeling. Advantageously, although the clusters are actually in higher dimensional space, they can be projected into 2 or 3 dimensional space and visualized.

Once the data from all of the samples to be analyzed have been collected, they are processed by a series of algorithms. These algorithms facilitate the binning of sample data (e.g., relative binding affinities) according to one or more data features. The computational process applied by a specific embodiment of the data clustering system can be divided into data collecting, similarity matrix calculation, sample clustering, and visualization stages

When the selected criteria comprises bind affinity for a panel of antibodies, the profiles obtained consist of conformational variants that have a similar binding affinity for each antibody in the panel. The selected criteria used to profile the sample may include any appropriate chemical, physical or functional characteristic. In a preferred embodiment, the criteria include the relative binding affinity for multiple antibodies. Some clusters may contain only one sample depending on the clustering method. It is postulated that the conformational variants within a profile have similar tertiary and secondary structures. Therefore, the conformational variants that are classified in the same profile may share other properties, such as antigenic properties and other properties.

The step of sorting conformational variants into clusters or profiles may be done manually or automatically using a suitable algorithm as discussed.

As mentioned above, it is preferred that the step of contacting the conformational variants with the antibodies is carried out in the presence of biological fluids and/or adjuvants since this enables protein antigens having a structure capable of binding to a neutralizing antibody in the presence of these compounds to be easily identified.

In one embodiment, the step of contacting the conformational variants is carried out with the protein antigen bound to a microparticle or solid support.

In another embodiment, the conformational variant protein antigen may be further tested for its ability to clear or partially clear neutralizing activity in serum in vitro. Suitable methods for determining neutralizing activity are known in the art. One method involves precipitation of neutralizing antibodies using antigens linked to beads, followed by determining the quantity of neutralizing antibody precipitated, possibly using a second antibody specific to the isotype of the neutralizing antibody or, alternatively, using a second antigen, such as protein A or protein G, with a greater affinity for the neutralizing antibody than the protein antigen. Protein antigen that is capable of clearing or partially clearing neutralizing activity is suitable for use in a pharmaceutical composition. Preferably the conformational variant protein antigen can clear neutralizing activity to a greater degree than the native conformation. The protein antigen may further be tested for its ability to raise neutralizing antibodies in vivo.

Preferred antigens are conformational variants that have a higher relative binding affinity for a neutralizing antibody compared to a non-neutralizing antibody. Preferred antigens are those that have the highest average or median affinity for a panel or plurality of neutralizing antibodies. Preferred antigens also are those antigens with the greatest difference between a neutralizing antibody or the average or median binding affinity for a panel of neutralizing antibodies and a non-neutralizing antibody or panel of non-neutralizing antibodies. The panel of antibodies may comprise at least 1, 2, 3, 4, 5, 6, 7, 8, 9, 10, 15, 20 or 25 neutralizing antibodies and 1, 2, 3, 4, 5, 6, 7, 8, 9, 10, 15, 20 or 25 non-neutralizing antibodies. Preferred antigens are conformational variant that have a higher increase in binding affinity for a neutralizing antibody than for a non-neutralizing antibodies, over the native conformation.

Production of protein antigen: The method of producing a pharmaceutical composition further comprises a step of producing the protein antigen. Because the protein antigen is a conformational variant or subset of the target protein, it will have already been produced by treating a sample of target protein with a particular set of conditions. Other conditions may be used to generate the same conformational variant(s). Accordingly, it is a simple matter to produce more protein antigen by treating a further sample of the target protein under the same set of conditions. Larger batches of the protein antigen could also be produced by scaling up the process. As discussed, the protein antigen may be comprised of multiple conformational variants. A sample may also be enriched for one or more conformational variants and methods can be optimized to produce batches initially enriched for one or more conformational variants.

Preferably, the protein antigen does not have the exact conformation of the native target antigen as determined by detailed structural analysis such as X-ray crystallography or NMR spectroscopy.

Binding of the protein antigen to a microparticle: The method for producing a composition of the present invention optionally comprises a step of binding the protein antigen (conformational variant) to a microparticle. Preferably, the composition is a pharmaceutical composition.

A microparticle is a solid or gel particle of up to 150 μm in diameter, more preferably about 100 nm to about 30 μm in diameter and most preferably about 500 nm to about 10 μm in diameter. Preferably the microparticle is of a diameter that permits parenteral administration without occluding needles and capillaries. It is also preferred that the microparticle is of a diameter that permits endocytosis by antigen presenting cells.

Microparticle size is readily determined by techniques well known in the art such as photon correlation spectroscopy, laser diffractometry (using a helium neon laser) and scanning electron microscopy.

In one aspect of the invention, the microparticles are preferably formed from pharmaceutically acceptable materials. These include inorganic materials such as metal salts. Microparticles formed from calcium phosphate or aluminum hydroxide are particularly preferred.

Methods for producing such microparticles are known in the art. For example, U.S. Pat. No. 6,355,271 discloses a method for producing calcium phosphate core particles having a diameter between 300 nm and 4000 nm. In summary, calcium phosphate core particles are prepared by mixing an aqueous solution of calcium chloride having a concentration between 5 mM and 100 mM with an aqueous solution of sodium citrate having a concentration of between 5 mM and 100 mM, followed by adding an aqueous solution of dibasic sodium phosphate having a concentration between 5 mM and 100 mM and mixing for 48 hours, or until a suitable particle size have been obtained.

In a further aspect of the invention, the microparticles are formed from polymeric materials. Suitable polymeric particles are known and include the cationic exchange resin powders disclosed in U.S. Pat. No. 5,942,242, many of which are commercially available.

In a preferred embodiment, the polymeric material is biodegradable. In this context, biodegradable means that the polymer will degrade in vivo to form smaller chemical species.

Suitable biodegradable polymers include a polyhydroxybutyric acid, a polycaprolactone, a polyothoester, a polyanhydride, a polyesteramide, a polyamino acid, a polycyanoacrylate, a polyamide, a polyacetal, a polyetherester, a polydioxanone, a polyalkene alkylate and a biodegradable polyurethane. In a particularly preferred embodiment, the biodegradable polymer is a poly (α-hydroxy acid). The poly (α-hydroxy acid) may be a polylactide, a polyglycolide, a poly(lactide-co-glycolide), poly (D,L-lactide-polyethylene glycol) or a poly(sulphobutyl-polyvinylalcohol)-g-(lactide-co-glycolide). Procedures for synthesizing these polymers are well known in the art. Many of these polymers are also commercially available from Boehringer Ingelheim.

Biodegradable polymeric microparticles may be prepared by methods standard in the art. One suitable method is the double emulsion solvent evaporation technique described in O'Hagan et al. (Vaccine (1993) 11:965-969) and Jeffrey et al. (Pharm. Res. (1993) 10:362). In this method, a polymer is combined with an organic solvent such as ethylacetate, dimethylchloride, acetonitrile, acetone or chloroform. The concentration of the polymer is in the range of 2-15%, and is more preferably about 4-10% and most preferably 6%. An approximately equal amount of an aqueous solution is added and the mixture is emulsified using, for example, a homogenizer. The emulsion is then combined with a larger volume of an aqueous solution of an emulsion stabilizer such as polyvinyl alcohol or polyvinyl pyrrolidone. The emulsion stabilizer is typically provided in about a 2-15% solution, more typically about a 4-10% solution. The mixture is then homogenized to produce a stable double emulsion. Organic solvents are then evaporated.

Microparticles can also be formed using spray drying and coacervation techniques. These are described in Thomasin et al. (J. Controlled Release (1996) 41:131), U.S. Pat. No. 2,800,457 and Masters, K. ((1976) Spray drying 2^{nd} Ed. Wiley N.Y.).

It will be apparent to one skilled in the art that the formulation parameters can be manipulated to allow the preparation of small (<5 μm) and large (>30 μm) microparticles. For example, reducing the speed and duration of agitation results in larger microparticles, as does increasing the volume of the aqueous phase. U.S. Pat. No. 6,565,777 discloses a method for producing polymeric particles of a size up to 10 μm in diameter. The method is susceptible to industrial scale application.

The protein antigen is bound to the surface of the microparticle. In this context, the word bound is intended to refer to a close association between the protein antigen and the surface of the microparticle. It encompasses the situation where the protein antigen is covalently attached to the microparticle as well as the situation where the antigen interacts with the surface of the antigen by means of non-covalent interactions such as hydrogen bonding, ionic bonding or hydrophobic interactions. Where the protein antigen interacts by means of non-covalent interactions, the antigen is described as being adsorbed to the surface of the microparticle.

In a preferred embodiment, the protein antigen is adsorbed to the surface of the microparticle. Adsorption may be achieved by simply by mixing the microparticles and protein antigen. Following adsorption, unbound protein antigen may be removed by filtration or centrifugation.

Where the microparticle is formed from a polymer, the protein antigen and microparticle may be covalently linked, following adsorption. Covalent bonds may be formed by treatment of the conformational variants of the target protein with a chemical cross-linking agent having two reactive groups, one reactive group recognizing a functional group on the protein antigen, and the other reactive group recognizing a functional group on the polymer. Suitable cross-linking agents, both cleavable and non-cleavable, are commercially available. Alternatively, a functional group on either the protein or antigen may be modified using standard chemical techniques prior to adsorption. Following adsorption, the modified functional group may be activated resulting in the formation of covalent cross-links.

Polymeric microparticles may additionally comprise a protein core composed of at least one protein molecule having the same primary amino acid sequence as the protein antigen. This protein molecule may or may not have the same conformation as the protein antigen bound to the surface of the microparticle.

Methods for entrapping protein molecules in polymeric microparticles (to generate a protein “core”) are known. Where polymeric microparticles are formed by the double emulsion solvent evaporation technique, an equal volume of an aqueous solution of the protein may be added to the polymer dissolved in the organic solvent. Since the protein molecules are contacted by the organic solvents, any secondary or tertiary structure may be lost. Accordingly, where it is desirable for the entrapped protein to have a neutralizing epitope, the conformation of the protein must be stabilized by intramolecular covalent crosslinking.

In a distinct embodiment, the protein molecules are not entrapped during formation of the microparticle. Instead, a protein core of 150 nm to 150 μm is generated as described in U.S. Pat. No. 6,534,064. In summary, protein particles are formed from purified protein by chemical precipitation, the use of cross-linking agents or by heat stabilization. The protein present in such a protein core may not retain any secondary or tertiary structure. Therefore, where it is desirable for the entrapped protein to have a neutralizing epitope, the conformation of the protein must be stabilized by intramolecular covalent crosslinking.

The protein antigen of the present invention may simply be adsorbed to the surface of the protein core. Alternatively, the protein antigen may be covalently linked to the microparticle following adsorption. Covalent cross links may be formed by a chemical cross linking agent. The crosslinking agent is not particularly limited and any agent capable of forming cross links in proteins may be used. Suitable chemical cross-linking agents include formaldehyde, glutaraldehyde, glyocal, malondialdehyde, succinialdehyde, adipaldehyde, phthalaldehyde, 4-Azidobenzoic acid (3-sulfo-N-succinimidyl) ester sodium salt, 1,4-Bis [3-(2-pyridyldithio) proprionamido]butane, Bis [2-(N-succinimidyl-oxocarbonyloxy)ethyl] sulfone, Bis-maleimidohexane, Bis-[2-(4-azidosalicylamido) ethyl] disulphide, 1,5-Difluoro-2,4-dinitrobenzene, Dimethyl pimelimidate, Dimethyl 3-3′-dithioproprionimidate dihydrochloride, Ethylene glycol disuccinate di(N-succinimidyl) ester, N-hydroxysuccinimidyl-4-azidosalicylic acid, and Sebacic acid bis(N-succinimidyl) ester. These chemical cross-linking agents are available from Sigma Aldrich and Pierce Biotechnology, Inc.

Covalent cross links may be also be formed by exposure to conditions that induce the formation of covalent cross links. Conditions that induce the formation of covalent cross links include oxidizing conditions, UV irradiation, and pH.

The protein core may also be surrounded by a pharmaceutically acceptable coating agent, that is releasable in vivo i.e. the coating agent must be removed in the body releasing the protein core. The coating agent must either therefore be soluble in water, or be degraded to smaller molecular species in vivo. The coating agent may be selected to enhance adsorption of the protein antigen to the microparticle.

In a preferred embodiment, the coating agent is calcium phosphate, cellobiose and polyethylene glycol. In a particularly preferred embodiment, the coating agent is a biodegradable polymer. Suitable biodegradable polymers are listed above. The protein antigen may be adsorbed or covalently linked to the coating agent.

Any microparticle for use in the present invention may also comprise an immunostimulatory molecule. This immunostimulatory molecule may be a molecule that enhances the cell mediated response. Alternatively, the immunostimulatory molecule may enhance the humoral response. Immunostimulatory molecules include cytokines (e.g. interleukins) and interferons.

Inmmunostimulatory molecules that enhance the cell mediated response include IL-2, IL-12, IL-18, interferon-α, interferon-β and interferon-γ.

Inmmunostimulatory molecules that enhance the humoral response include IL-4, IL-5, IL-6, IL-10 and IL-13.

The immunostimulatory molecule may be adsorbed to the surface of the microparticle or entrapped in the microparticle in the same way described for the protein antigen.

Where microparticles are composed of a biodegradable polymer, one skilled in the art will be able to control the rate of degradation. The rate of degradation is dependent upon the combination of polymers forming the microparticle, and the molecular weight of these polymers. Also, where co-polymers are used, the ratio of the monomers is known to affect degradation rate. For example, a poly(lactide-co-glycolide) polymer having a lactide:glycolide ratio of 50:50 degrades more rapidly than a polymer having a lactide:glycolide ratio of 75:25. A polymer having a lactide:glycolide ratio of 90:10 degrades even more slowly.

One skilled in the art will be able to select suitable microparticles for each application. By selecting a microparticle containing entrapped protein antigen that degrades slowly, a slow release phamaceutical composition is generated. The sustained release of antigen generated from such a pharmaceutical composition may result in a sufficiently high immune response to not require further booster injections.

The pharmaceutical compositions of the present invention (comprising protein antigen adsorbed to a microparticle) generate a strong humoral immune response. Without being bound by theory, it is hypothesized that this is because both the T-cell dependent and T-cell independent pathways of the humoral response are stimulated by the pharmaceutical composition. First, the T-cell independent pathway is stimulated because the microparticle is coated with multiple copies of the same antigen and therefore resembles a T-cell independent antigen. This results in a B-cell activation and antibody production by a T-cell independent mechanism.

Second, the microparticles are suitable for endocytosis by heterogeneous virgin B-cells expressing antibodies recognizing the protein antigen. This contributes to the activation of the B-cells by a T-cell dependent pathway. Activated B cells proliferate and differentiate into antibody-secreting plasma cells.

Furthermore, the microparticles will also be endocytosed by antigen presenting cells. The antigen presenting cells then activate helper T cells, which then help activate B cells leading to an increased humoral response. The helper T cells also activate cytotoxic T cells, leading to an increased cell mediated response.

Compositions: The present invention also provides for compositions, including pharmaceutical compositions, prepared according to the method described herein. Accordingly the invention provides a composition comprising a protein antigen obtained by treatment of a plurality of isolated samples of a target protein under different conditions to produce a plurality of isolated conformational variants. The conformational variant samples may be clustered based on their binding profiles against a panel of antibodies or other selected criteria. The samples, before or after clustering, or one or more representative sample from one or more clusters are tested for their ability to bind one or more neutralizing antibodies, their ability to clear neutralizing activity, their ability to raise neutralizing antibodies or another method used to predict whether the sample can raise neutralizing antibodies in vivo. The samples may further be assayed to compare their selective binding affinity to neutralizing versus non-neutralizing antibodies. Normally, samples that preferentially bind neutralizing antibodies over non-neutralizing antibodies or preferentially bind neutralizing antibodies over the native confirmation are preferred.

In a further aspect, the invention provides a composition comprising a protein antigen capable of binding a neutralizing antibody with higher affinity than a non-neutralizing antibody (both of which are specific for the same protein or even the same epitope of a protein), which protein antigen is not present in a fully native conformation. The conformationally trapped protein antigen may further include or be bound to a microparticle having a diameter of up to 150 μm.

The composition may be a composition capable of raising an immune response against the organism from which the antigen is from. Preferably, the immune response comprises the production of neutralizing antibodies. More specifically, the composition may be a vaccine. That is, where the target protein was selected to be an antigen present on or produced by (e.g., a toxin or virulence) an invading microorganism, the composition may be suitable for immunizing or partially immunizing a subject against that microorganism.

The invention also provides compositions, including vaccines, comprising a plurality of protein antigens. In a preferred embodiment, each protein antigen present is a conformational variant that binds to a neutralizing antibody. The protein antigens may be from the same or different conformational variant sample or conformational variant sample cluster. Further, the protein antigens may be different epitopes, from different portions of a protein, different proteins or proteins from different organisms. In one embodiment, the protein antigens bind to different neutralizing antibodies. Such a composition may be suitable for the immunization of a subject against a single or plurality of infectious diseases.

Advantageously, this invention provides pharmaceutical compositions, including vaccines, that immunize or partially immunize an animal against multiple infectious diseases likely to be encountered in certain regions or periods throughout one's lifetime. Such antigens are suitable for immunizing people indigenous or travelers to such regions. In addition, the invention provides compositions that immunize against many of the infectious diseases foreseen to be used by bioterrorists. Such compositions including vaccines are useful for immunizing the armed forces and the civilian population. In addition, the invention provides for the enhancement of the generation of compositions useful for raising an immune response to an antigen or organism including vaccines.

Where the composition is a vaccine, the composition may further comprise an adjuvant. Any adjuvant capable of enhancing an immune response to the antigen may be used. Suitable adjuvants include oil-in-water emulsions, liposomes, squalene mixtures, muramyl peptide preparations, saponin derivatives, mycobacterium cell wall preparations, monophosphoryl lipid A, mycolic acid derivatives, non-ionic block surfactants, Quil A and cholera toxin B subunit. Preferably, the adjuvant is pharmaceutically acceptable to humans. Examples of adjuvants that are pharmaceutically acceptable to humans include aluminium hydroxide, aluminium potassium sulfate, QS-21 (a saponin product derived from the soapbark tree, Quillaja saponaria) and CAP (BioSante Pharmaceuticals Inc.).

The compositions of the present invention may further comprise an immunostimulatory agent including those disclosed herein.

Preferably, the compositions are pharmaceutical compositions.

Pharmaceutical Composition Development: Suitable target proteins for pharmaceutical composition development include proteins from a pathogenic microorganism. Conformational variants generated from such a target protein are clustered or profiled using a panel of antibodies capable of binding to the protein derived from the organism or other selected criteria. This generates several clusters of conformational variants with the same or similar profile. Profiles comprising conformational variants that bind to a neutralizing antibody, that clear neutralizing activity or raise neutralizing antibodies may be further screened to identify suitable protein antigens for use in a pharmaceutical composition. Alternatively, samples may be screened with one or more neutralizing antibodies before clustering to limit the number of samples tested with a panel of antibodies or other methods of measuring selected criteria.

The conformational variant samples from selected clusters may be tested for their ability to clear neutralizing activity from sera of individuals vaccinated, exposed or infected with the pathogen in question. Typically, following incubation of the sera with a known quantity of the conformational variant, the sera is assayed for the neutralizing activity remaining. Several methods determining the neutralizing activity are known, including precipitation of neutralizing antibodies with antigen attached to beads and determination of the quantity of neutralizing antibody precipitated.

The conformational variants may be tested for their stability in various biological fluids, in the presence of various adjuvant, at various doses.

Those conformational variants that bind to a neutralizing antibody, which are able to clear neutralizing activity and which are stable in the presence of biological fluids, adjuvants and at the required dosage are suitable protein antigens for inclusion in a pharmaceutical composition. They may, for example, be used in compositions, including vaccines, to stimulate an immune response or to immunize or partially immunize subjects against the pathogen from which the target protein was derived.

Where no neutralizing antibody is known, this invention provides a method of screening for compositions (e.g., vaccines) that generate neutralizing antibodies to a protein of a pathogen. First, conformational variants of the protein from the pathogen are profiled using non-neutralizing antibodies that recognize the target protein or other selected criteria. The structures of conformational variants cluster may then be analyzed as described. If this analysis reveals that a cluster is composed of conformational variants having distinct secondary or tertiary structures, the cluster is subdivided accordingly.

Representative members of each cluster may then be injected into host animals (typically mice, rats or rabbits) to raise antibodies against the conformational variants according to methods that are standard in the art. The antibodies raised are then tested for neutralizing activity using standard methods. Those conformational variants that generate antibodies having neutralizing activity are suitable for pharmaceutical composition development and other uses. They may then be tested for their stability in biological fluids, in the presence of pharmaceutically acceptable adjuvants and at the required dosage as described above to identify a suitable conformational variant for use in a pharmaceutical or other composition.

Alternatively, samples may be tested for their ability to raise neutralizing antibodies by challenging treated animals with the pathogen from which the protein antigen was derived and measuring resistance to or virulence of the pathogen.

Preferably, the protein antigens are tested for their ability to bind to neutralizing antibodies when bound to a microparticle adjuvant. The use of such an adjuvant is particularly preferred and the composition of the microparticle may be selected to have particular characteristics. For example, if it is desirable for the protein antigen to be released slowly over a long period of time (up to one month) a biodegradable polymeric particle which degrades in vivo over this time frame should be selected. Where the pharmaceutical composition is a vaccine, or other composition useful for stimulating an immune response such a formulation may result in a vaccine for which no booster injections are required.

The compositions of the invention may be administered to a vertebrate subject (e.g., human, dog, cat, cow, horse, pig, etc.) to elicit an immune response. Preferably, the subject is a mammal and it is particularly preferred that the subject is a human subject. The compositions may be administered using methods known in the art. The compositions, for example, may be injected either subcutaneously, intraperitoneally, intradermally, intravenously or intramuscularly. The dosage regimen will at least in part be determined by the needs of the subject and an appropriate regimen will be clear to a medical practitioner or others skilled in the art. For example, the antibody titre in the serum of the subject may be monitored and booster doses given following a reduction in this titre. Typically, one to ten separate doses are given at intervals of one to four months.

A summary of an example of a method according to the invention is provided below (in the form of a flow-chart).

Obtain a protein of interest

↓

Denature target protein or begin with target protein in the native state

↓

Re-fold or unfold the target protein in various conditions/times under reducing conditions to induce multiple conformations (each sample has a different refolding or unfolding condition)

↓

Stabilize the protein conformations, for example, by adjusting conditions to induce covalent crosslinking (reducing conditions→oxidizing, denaturing conditions→refolding)

↓

Isolate stabilized species via chromatography or other methods

↓

Profile each isolated stable species using a panel of monoclonal antibodies, phage display clones, or other selected criteria

↓

Cluster samples with similar proteins

↓

Test representative variants from each cluster to identify conditions that have higher affinity for neutralizing activity

↓

Further test high affinity conformations or subsets to determine whether they can clear neutralizing activity from sera

↓

Those variants or conformational subsets that have both high affinity and are able to clear neutralizing activity represent potential antigens

↓

Assay conditions that stabilize structure in presence of adjuvant, in dosage fomulation, and in biological fluids

### EXAMPLE 1

### Preparation of Conformational Variants

The principal biophysical techniques employed is this example were circular dichroism and fluorescence thereby allowing for extensive characterization of structural changes. The protein studied was bovine pancreatic trypsin inhibitor. For illustrative purposes, a limited test bed of conditions were explored comprising 28 unique formulations and an excess of 350 samples. The solution space included unary and binary mixtures of (de)stabilizers. Detected intermediates may be stabilized through careful control of solution conditions as well as by means of chemical cross linkers. In this particular case, the folding reaction was monitored through the introduction of sulfhydryl directed cross linking agents. Individual species were then isolated using HPLC chromatography and subjected to further characterization including circular dichroism spectroscopy, fluorescence spectroscopy, mass spectroscopy, and peptide mapping.

In one embodiment the invention involves a step of finding conditions which stabilize equilibrium intermediate(s), broaden the unfolding reaction transition region, or uncouple the cooperativity of the unfolding reaction. Once identified, these conditions are used for the cross linking reaction to stabilize the conformational variants.

A concentrated stock solution (25-fold) of bovine pancreatic trypsin inhibitor, BPTI, (Swiss-Prot primary accession number P00974; EMBL accession number X03365) was prepared by dissolving lyophilized protein in buffer (10 mM phosphate, 0.2mM EDTA, pH 7.6). BPTI is also known as aprotinin. The protein stock solution was evenly dispensed into an array of samples. An array was typically 15 samples and each sample in the array contained 3.5 μM BPTI, 10 mM phosphate, 0.2 mM EDTA, pH 7.6 but a variable amount of denaturant. Guanidine hydrochloride was used as the denaturant and its concentration ranged from 0 to 5 M. Equal amounts of an additive were dispensed into each sample of the array. Suitable additives include: reducing agents, oxidizing agents, alcohols, organic solvents, inorganic solvents, detergents, osmolytes, acids, and bases. Dithiotheitol, a reducing agent, was used. For example, one array of samples would contain 1 mM dithriothreitol and another array of samples would contain 5 mM dithriothreitol. A second additive was added to an array of samples. For example, an array of samples would contain 1 mM dithriothreitol and 5% trifluoroethanol.

All samples were equilibrated overnight at 25° C. Far-UV CD spectra of each sample were collected on a spectropolarimeter meter equipped with a thermoelectric cell holder a high-throughput autosampler accessory. Tryptophan and/or tyrosine fluorescence data are also collected using a spectrofluorometer equipped with a thermoelectric cell holder and a high-throughput autosampler.

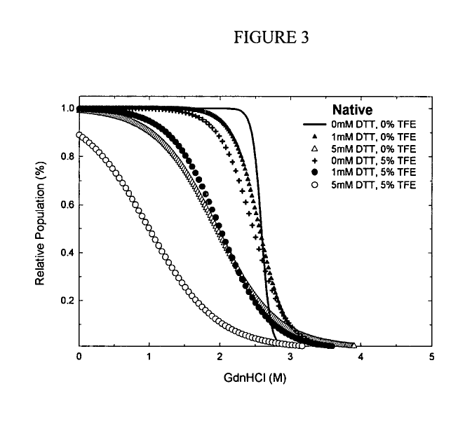

The spectra for each array were clustered and analyzed according to equations standard in the art by fitting to two-, three-, or four-state models via nonlinear least squares methods to determine BPTI's thermodynamics (i.e., stability) under those particular solution conditions. The thermodynamics were then used to recast the data into population distributions with respect to denaturant (FIG. 3). The relative populations of each species as a function of guanidine hydrochloride were calculated by converting the thermodynamic parameters from the three-state fit into equilibrium constants for each transition. All data analysis was standard for the art. For example, a three-state equilibrium unfolding model is described by the following equation:

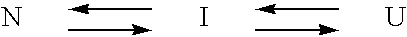

where N, I, and U are the native, intermediate, and denatured states, respectively. The equilibrium constants between each species are K_{NI}=[I]/[N] and K_{IU}=[U]/[I]. The denaturation profile can be cast in terms of the apparent fraction of unfolded protein, Fₐₚₚ, by:

Fₐₚₚ=(Y_{N}−Y_{obs})/(Y_{N}−Y_{U})

where Y_{obs} is the observed signal at a given denaturant concentrations and Y_{N} and Y_{U} are the respective signals of the native and unfolded protein. Y_{N} and Y_{U} were estimated by linear extrapolation of the data in the pre-and post-transition regions, respectively.

The thermodynamic parameters are contained within the rewritten expression for the apparent fraction of unfolded protein:

Fₐₚₚ=(Z_{I}K_{NI}+K_{NU})/(1+K_{NI}+K_{NU})

where

Z_{I}=(Y_{I}−Y_{N})/(Y_{U}−Y_{N})

and

K_{NU}=K_{NI}K_{IU}.

The parameter Z_{I} is an approximation representing the fraction of unfolded character displayed by the intermediate. A value of zero or one indicates that the intermediate resembles either the native or unfolded state, respectively.

A linear dependence of the apparent free energy difference on the denaturant concentration was assumed in relating the equilibrium constants to free energy changes.

$$\begin{matrix}Δ G° = Δ G° ( {H}_{2} O ) + m [ urea ] \\ = - RT ln K\end{matrix}$$

where ΔG° and ΔG°(H₂O) are the Gibbs free energy changes in the presence or absence of a given urea concentration, respectively. The sensitivity of the apparent free energy change to denaturant concentration is represented by m.

Cross linking: A concentrated protein stock solution (10 to 25-fold) was prepared by dissolving lyophilized protein in optimized solution conditions identified from the previous equilibrium studies. For example, 2.44 M guanidine hydrochloride, 5 mM dithriothreitol, 10 mM phosphate, 0.2 mM EDTA, pH 7.5. The protein stock solution was allowed to equilibrate overnight at 25° C.

The stock solution of unfolded protein was diluted 10 to 25-fold with buffer (10 mM phosphate, 0.2 mM EDTA, pH 7.5) containing oxidizing agent (10 mM oxidized dithiothreitol final concentration). A covalent cross linker or mixture of cross linkers were added to the refolding protein at various time increments (in this case: 0.1, 1, 2, 3, 4, 6, 8, 10, 12, 14, 16, 20, 30, 60 minutes) to a final cross linker to protein ratio of 10-25:1.

Homo- and heterobifunctional cross linkers targeting amines, sulfhydryls, carbohydrates, carboxyls, and hydroxyls can be used to cross link the protein. Nonspecific cross linkers can also be used. We chose to focus on two homobifunctional and two heterobifunctional cross linkers. The homobifunctional cross linkers used were BMOE and BM[PEO]₃ and are specific to sulhydryls. Sulfo-EMCS and Sulfo-SMCC were the heterobifunctional cross linkers used and target amines and sulfhydryls. Concentrated stock of the cross linkers (—10 mM) were prepared according to the manufacturer's instructions.

The cross linking reaction took place for 15 minutes at 25° C. before quenching with iodoacetic acid (final concentration of 0.1 M). Alternatively, iodoacetamide can be used as the quenching agent. The quenched samples were dialyzed twice against 100 mL of buffer (10 mM phosphate, 0.2 mM EDTA, pH 7.5) for 30 minutes to remove excess cross linker, denaturant, and salts. A PIERCE MICRODIALYZER SYSTEM 100 was used to dialyse the samples. The dialyzed samples were stored at 25° C.

The refolding time-point samples were analyzed by HPLC using a cation exchange column in a 20 mM phosphate buffer system (pH 6.2) and a salt gradient of 0-0.5M NaCi with a 1.0 mL/min flow rate (FIG. 2). Fractions were collected every minute. Peaks of similar retention times were pooled and concentrated using Centricon YM-3 spin concentrators (MWCO 3000) to a final volume of 1 mL. The concentrated samples were analyzed by standard and peptide mapping mass spectroscopy techniques to determine the extent of and identify the sites of covalent cross linking.

Affinity Measurements: The relative affinities of the isolated species against a test bed of antibodies are determined using standard ELISA and surface plasmon resonance techniques. Data analyses are performed by methods typical of one skilled in the art.

Whilst the invention has been described in terms of preferred embodiments, the skilled artisan will appreciate that various modifications, substitutions, omissions and changes may be made without departing from the spirit thereof. Accordingly, it is intended that the scope of the present invention be limited solely by the scope of the following claims, including equivalents thereof.

1. A method of producing a composition comprising a protein antigen, said method comprising the steps of:
a) providing a plurality of samples comprising a protein, said samples differing with respect to the conformation state of said protein;
b) identifying a sample from said plurality of samples that comprises a conformational variant of said protein capable of stimulating the production of neutralizing antibodies, against a pathogen from which said protein was derived.

2. The method of claim 1, wherein said conformational variants have the same primary amino acid sequence, but differ with respect to their secondary or tertiary structure.

3. The method of claim 1, wherein said method comprises the step of clustering samples based on a profile of selected criteria.

4. The method of claim 3, wherein said criteria comprises the ability to bind or the affinity for an antibody, or antigen binding fragment thereof, or plurality of antibodies specific for said protein.

5. The method of claim 4, wherein said antibody is:
a) a non-neutralizing antibody;
b) a neutralizing antibody;
c) a polyclonal antibody;
d) a monoclonal antibody;
e) contacted with said protein in the presence of biological fluids.

6. The method of claim 1, wherein:
a) said conformational variants are obtained by treating said protein in a native conformation under different conditions;
b) said conformational variants are obtained by treating a sample of protein having a native conformation under different conditions;
c) said conformational variants are obtained by treating a sample of denatured or partially denatured protein under different conditions; or
d) a secondary or tertiary structure of said conformational variant is stabilized.

7. The method of claim 1, wherein said identifying comprises identifying a sample comprising a protein conformational variant that:
a) binds to a neutralizing antibody;
b) binds to one or more neutralizing antibodies with a higher relative binding affinity than one or more non-neutralizing antibodies;
c) partially clears or clears neutralizing activity in serum in vitro;
d) stimulates the production of neutralizing antibodies in vivo;
e) binds a neutralizing antibody with a higher than the neutralizing antibody binds the native conformation;
f) wherein the affinity of a neutralizing antibody for the conformational variant is greater than the affinity of the neutralizing antibody for the native conformation;
g) wherein the increase in affinity of f) is greater than an increase in affinity of a non-neutralizing antibody for the conformational variant over the native conformation;
h) wherein the affinity of a neutralizing antibody for the conformational variant is greater than the affinity of the neutralizing antibody for a native conformation, but wherein there is no difference or a decrease in affinity of a non-neutralizing antibody for the conformational variant over the native conformation.

8. The method of claim 1, wherein said protein is structurally characterized using method selected from the group consisting of:
circular dichroism spectropolarimetry, fluorescence spectroscopy using either intrinsic or extrinsic fluorescent probes, mass spectroscopy, UV-VIS spectroscopy, NMR, small angle X-ray scattering, enzymatic activity, ion exchange, hydrophobic interaction, reverse phase chromatography, gel filtration and affinity.

9. The method of claim 6, wherein said conformational variant is stabilized using a covalent linker.

10. The method of claim 9, wherein covalent linker targets an amino group, a carboxyl group, a hydroxyl group, a carbohydrate or a glutaraldehyde.

11. The method of claim 9, wherein said linker target is created by in vitro mutagenesis of the amino acid sequence or a nucleic acid sequence encoding said protein.

12. The method of claim 1, wherein a purification step enriches a sample for a conformational variant.

13. The method of claim 12, wherein said sample is enriched for a conformational variant using ion exchange, chromatography, hydrophobic interaction chromatography, reverse phase chromatography, gel filtration chromatography or affinity chromatography.

14. The method of claim 12, wherein said samples are enriched using one or more neutralizing antibodies.

15. The method of claim 4, wherein said affinity is measured using a method selected from the group consisting of: ELISA, surface plasma resonance, gel mobility shift assay, isothermal titration calorimetry equilibrium dialysis, centrifugation and fluorescent resonant energy transfer.

16. A method according to claim 1, which further comprises contacting each conformational variant sample with a non-neutralizing and a neutralizing antibody and identifying a sample comprising a conformational variant or subset that binds to the neutralizing antibody with higher affinity than to the non-neutralizing antibody.

17. A method according to claim 1, wherein said conformational variant is bound to a microparticle having a diameter up to 150 μm.

18. A method according to claim 17, wherein said microparticle comprises biodegradable polymer or other biodegradable microparticle material.

19. A method according to claim 17, wherein:
(a) said microparticle comprises said conformational variant bound to its outer surface;
(b) the microparticle core is surrounded by a pharmaceutically acceptable coating agent, that is releasable in vivo;
(c) said coating agent of (b) is selected from the group consisting of a biodegradable polymer, calcium phosphate, cellobiose and polyethylene glycol;
(d) said biodegradable polymer of (c) is selected from the group consisting of a polylactide, a polyglycolide, a poly(lactide-co-glycolide), poly (D,L-lactide-polyethylene glycol), a poly(sulphobutyl-polyvinylalcohol)-g-(lactide-co-glycolide), a polyhydroxybutyric acid, a polycaprolactone, a polyothoester, a polyanhydride, a polyesteramide, a polyamino acid, a polycyanoacrylate, a polyamide, a polyacetal, a polyetherester, a polydioxanone, a polyalkene alkylate and a biodegradable polyurethane;
(e) the microparticle comprises a pharmaceutically acceptable biodegradable polymer;
(f) said biodegradable polymer of (e) is selected from the group consisting of a polylactide, a polyglycolide, a poly(lactide-co-glycolide), poly (D,L-lactide-polyethylene glycol), a poly(sulphobutyl-polyvinylalcohol)-g-(lactide-co-glycolide), a polyhydroxybutyric acid, a polycaprolactone, a polyothoester, a polyanhydride, a polyesteramide, a polyamino acid, a polycyanoacrylate, a polyamide, a polyacetal, a polyetherester, a polydioxanone, a polyalkene alkylate and a biodegradable polyurethane;
(g) the microparticle comprises a metal salt;
(h) said metal salt of (g) is calcium hydroxide or aluminium hydroxide;
(i) the method further comprises a step of covalently linking the protein antigen to the surface of the microparticle;
(j) said microparticle further comprises an immunostimulatory molecule;
(k) wherein said immunostimulatory molecule of (j) is a molecule that results in an elevated humoral response;
(l) wherein said immunostimulatory molecule of (k) is IL-4, IL-5, IL-6, IL-10 and IL-13.

20. A pharmaceutical composition comprising a conformational variant obtained by the method of claim 1.

21. A pharmaceutical composition according to claim 20, wherein the conformational variant is a protein antigen that binds to a neutralizing antibody with a higher affinity than a non-neutralizing antibody.

22. A pharmaceutical composition according to claim 20, wherein the structure of the conformational variants is stabilized.

23. A pharmaceutical composition according to claim 1, wherein the conformational variant protein antigen is capable of partially clearing or clearing neutralizing activity in serum in vitro.

24. The pharmaceutical composition of claim 20, further comprising a microparticle.

25. A pharmaceutical composition according to claim 24, wherein:
(a) said microparticle comprises a core comprising at least one protein molecule that comprises the same primary amino acid sequence as the protein antigen;
(b) said microparticle comprises a protein core composed of at least one protein molecule having greater than 95% amino acid sequence identity as the protein antigen;
(c) the protein core of (a) is surrounded by a pharmaceutically acceptable coating agent, that is releasable in vivo;
(d) said coating agent of (c) is selected from the group consisting of a biodegradable polymer, calcium phosphate, cellobiose and polyethylene glycol;
(e) said biodegradable polymer of (d) is selected from the group consisting of a polylactide, a polyglycolide, a poly(lactide-co-glycolide), poly (D,L-lactide-polyethylene glycol), a poly(sulphobutyl-polyvinylalcohol)-g-(lactide-co-glycolide), a polyhydroxybutyric acid, a polycaprolactone, a polyothoester, a polyanhydride, a polyesteramide, a polyamino acid, a polycyanoacrylate, a polyamide, a polyacetal, a polyetherester, a polydioxanone, a polyalkene alkylate and a biodegradable polyurethane;
(f) the microparticle comprises a pharmaceutically acceptable biodegradable polymer;
(g) said biodegradable polymer of (f) is selected from the group consisting of a polylactide, a polyglycolide, a poly(lactide-co-glycolide), poly (D,L-lactide-polyethylene glycol), a poly(sulphobutyl-polyvinylalcohol)-g-(lactide-co-glycolide), a polyhydroxybutyric acid, a polycaprolactone, a polyothoester, a polyanhydride, a polyesteramide, a polyamino acid, a polycyanoacrylate, a polyamide, a polyacetal, a polyetherester, a polydioxanone, a polyalkene alkylate and a biodegradable polyurethane;
(h) the microparticle comprises a metal salt;
(i) said metal salt of (h) is calcium hydroxide or aluminium hydroxide;
(j) the protein antigen is covalently bound to the surface of the microparticle.

26. A pharmaceutical composition according to claim 20, wherein said composition further comprises an immunostimulatory molecule.

27. A pharmaceutical composition according to claim 26, wherein said immunostimulatory molecule is a molecule that results in an elevated humoral response.

28. A pharmaceutical composition according to claim 27, wherein said immunostimulatory molecule is IL-4, IL-5, IL-10 and IL-13.

29. A pharmaceutical composition according to claim 20:
a) suitable for causing an immune response in for immunization of a subject against a pathogen;
b) which further comprises a second protein antigen;
c) which further comprises an adjuvant; or
d) suitable for partially or fully immunizing a subject against a pathogen from which the conformational variant was derived.

30. The pharmaceutical composition of claim 29, wherein said pathogen is a:
a) virus;
b) bacteria; or
c) fungus.
